(19) United States
(12) Patent Application Publication
(10) Pub. No.: US 2025/0029715 A1
(43) Pub. Date: Jan. 23, 2025

(54) SYSTEMS AND METHODS FOR EXTRACTING DIAGNOSTIC AND RESOLUTION PROCEDURES FROM HETEROGENOUS INFORMATION SOURCES

(71) Applicant: KONINKLIJKE PHILIPS N.V., EINDHOVEN (NL)

(72) Inventors: Milosh STOLIKJ, EINDHOVEN (NL); Lu WANG, EINDHOVEN (NL); Qi GAO, EINDHOVEN (NL)

(21) Appl. No.: 18/706,799

(22) PCT Filed: Nov. 1, 2022
(86) PCT No.: PCT/EP2022/080454

§ 371 Date: May 2, 2024

Related U.S. Application Data
(60) Provisional application No. 63/277,377, filed on Nov. 9, 2021.

(30) Foreign Application Priority Data
Mar. 18, 2022 (EP) 22162940.5

(51) Int. Cl. G16H 40/40

(52) U.S. Cl. CPC G16H 40/40

(57) ABSTRACT

A non-transitory computer readable medium (26) stores instructions executable by at least one electronic processor (20) to perform a medical device servicing method (100) for a medical device (12), including: deriving a representation (36) of abase diagnostic procedure for diagnosing a malfunction of the medical device from one or more service manuals (32); analyzing historical service records (34) to extract diagnostic procedures applied in historical service cases for diagnosing the malfunction of the medical device; adding the extracted diagnostic procedures to the representation of the base diagnostic procedure to generate an updated representation (40) of the base diagnostic procedure; calculating likelihood of success scores (42) for each path of the updated representation of the base diagnostic procedure based on statistics of outcomes of the historical service cases; and outputting the updated representation of the base diagnostic procedure on an electronic processing device (18) operable by a service engineer (SE).

### FIELD OF THE INVENTION

The following relates generally to the medical imaging arts, medical imaging device maintenance arts, medical imaging device diagnostic procedure arts, and related arts.

### BACKGROUND OF THE INVENTION

A medical imaging system or other medical device may occasionally malfunction, and thus fail to work properly. The malfunction is recognized by an operator of the medical device based on observed symptoms, such as failure of the medical device to operate, unsatisfactory output of the medical device (e.g., a patient monitor unable to read a vital sign sensor, a medical imaging device producing low-quality images, or so forth), and/or by an error code produced by the medical device, or based on other symptoms. Depending on the malfunction, the problem can sometimes be resolved remotely, by a remote service engineer (RSE). During diagnosis, the RSE can interact with several tools that can aid during system troubleshooting, and for identifying the best course of action to resolve the problem. For example, the RSE may decide to perform several actions, such as searching through historical service records to find similar service cases, and identify how they were resolved, searching through technical documentation for possible root causes and potential solutions, inspecting the log files of the medical imaging system, to identify relevant diagnostic information such as error messages, contacting the customer to get a description of the problem, and so forth.

After the troubleshooting, the RSE should be able to determine the root cause of the problem and identify the right service solution. The service solution can be executed remotely (e.g., a software update, remote calibration, etc.) or on-site when some spare parts need to be replaced.

However, RSEs are often under time pressure to perform the troubleshooting and decide on the most appropriate service action. To make an informed decision, RSEs need to collect and examine relevant information from various sources. Among the plethora of sources, RSEs may access technical documentation, such as service manuals, installation manuals etc., which list potential symptoms, sequences of steps to perform diagnosis, and instructions on which service actions to take. However, as this is static documentation, it may not be complete, or it may not be up to date. Similarly, RSEs may access historical service records, to inspect how the same (or similar) problem was successfully resolved in the past by other RSEs. However, due to the complexity of problem and medical system as well as the amount information reported in both historical service records and technical documentation, it is challenging for the RSEs to quickly identify the most relevant information for the current problem and system configuration.

The following discloses certain improvements to overcome these problems and others.

### SUMMARY OF THE INVENTION

In some embodiments disclosed herein, a non-transitory computer readable medium stores instructions executable by at least one electronic processor to perform a medical device servicing method for a medical device, including: deriving a representation of a base diagnostic procedure for diagnosing a malfunction of the medical device from one or more service manuals; analyzing historical service records to extract diagnostic procedures applied in historical service cases for diagnosing the malfunction of the medical device; adding the extracted diagnostic procedures to the representation of the base diagnostic procedure to generate an updated representation of the base diagnostic procedure; calculating likelihood of success scores for each path of the updated representation of the base diagnostic procedure based on statistics of outcomes of the historical service cases; and outputting the updated representation of the base diagnostic procedure on an electronic processing device operable by a service engineer (SE).

In some embodiments disclosed herein, an apparatus includes a display device, and at least one electronic processor programmed to: augment a representation of a base diagnostic procedure for diagnosing a malfunction of the medical device with diagnostic procedures applied in historical service cases for diagnosing the malfunction of the medical device to generate an updated representation of the base diagnostic procedure; calculate likelihood of success scores for each path of the updated representation of the base diagnostic procedure based on statistics of outcomes of the historical service cases; and output the updated representation of the base diagnostic procedure on the display device including at least a portion of the paths of the updated representation annotated by the calculated likelihood of success scores.

In some embodiments disclosed herein, a medical device servicing method includes: analyzing historical service records to extract diagnostic procedures applied in historical service cases for diagnosing a malfunction of the medical device; combining the extracted diagnostic procedures to generate a fault tree for diagnosing the malfunction of the medical device; labelling each path of the fault tree with a label comprising a number of historical service cases resolved by that path or with a likelihood of success score calculated based on the number of historical service cases resolved by that path, wherein each path comprises one or more routes or procedures performed by a service engineer for diagnosing a malfunction of the medical device; and outputting a representation of the fault tree including the labels.

One advantage resides in supporting the servicing of a medical device by combining information from technical documentation and historical service records into a single diagnostic procedure view for display.

Another advantage resides in supporting the servicing of a medical device by building a diagnostic and resolution procedure from information collected from service manuals augmented with findings from historical service records.

Another advantage resides in enabling a service engineer to quickly judge the different diagnostic paths that could be taken, the potential service actions, and the likelihood of each path occurring and successfully resolving the problem, based on historical data.

Another advantage resides in providing a view of a combination of information from technical documentation and historical service records created interactively by service engineers, allowing the service engineers to request details of certain actions, and record the actions they performed.

Another advantage resides in automatically generating a service record report with a summary and logged interactions of a service engineer during a servicing process.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a medical device servicing apparatus in accordance with the present disclosure.

FIG. 2 diagrammatically illustrates medical device servicing method using the apparatus of FIG. 1.

FIGS. 3-7 show examples outputs of the apparatus of FIG. 1.

### DETAILED DESCRIPTION OF EMBODIMENTS

The following relates to providing an improved diagnostic procedure recommender system based on published service manuals and historical service records.

Initially, a base diagnostic procedure for diagnosing the malfunction of the medical device is provided electronically, or is extracted by natural language processing (NLP) or computer vision (CV) from a published service manual. The service manual may, for example, be in binary (e.g., PDF) format, or in a structured machine-readable format. For the binary format manuals, optical character recognition (OCR) and NLP are applied to extract diagnostic procedure information from text, while these tools along with CV are applied to extract diagnostic procedure information from flowcharts or other diagrams. In one approach, parts of the manual are classified by subsystem, error code, and/or other classification schema to facilitate retrieving relevant parts for specific diagnostic tasks. The base diagnostic procedure is suitably represented as a diagnostic tree, although other representations such as a diagnostic map are also contemplated. Alternatively, in some other embodiments the base diagnostic may be provided directly, e.g. as a data structure representing the base diagnostic procedure as fault tree, or may be omitted entirely.

Next, historical service records are analyzed by NLP and/or based on structure of the service reports to extract diagnostic procedures that were actually applied by service engineers (SEs) in diagnosing the malfunction of the medical device under consideration. The extracted diagnostic procedures are expected to include numerous instances of the base diagnostic procedure outlined in the service manual, but may also include variant diagnostic procedures or even wholly new diagnostic procedures developed by service engineers to resolve specific historical cases. Statistics are also developed on the number of historical cases resolved by each of those diagnostic procedures.

The base diagnostic procedure is then augmented by additional paths corresponding to other diagnostic procedures (besides the base procedure) that were extracted from the historical service records, and each path (including both those of the base procedure and any variant or wholly different procedures extracted from the historical records) is labeled with number of historical cases resolved by that path. This produces an augmented diagnostic procedure with typically more paths than the base diagnostic procedure. As used herein, the term “path” (and variants thereof) refers to one or more routes or procedures performed by a service engineer for diagnosing a malfunction of a medical device. In other embodiments, the base diagnostic procedure may be omitted entirely, and the extracted diagnostic procedures combined to generate a fault tree for diagnosing the malfunction of the medical device, which serves in place of the augmented diagnostic procedure in the subsequent steps.

To apply the augmented diagnostic procedure in a current case, likelihood of success scores are assigned to the various paths of the procedure. An example scoring formula for a path comprises a weighted sum of factors including: (i) the number of cases resolved by that path; (ii) resolution quality, e.g., time to solution or customer satisfaction feedback; and (iii) similarity of the path to the particulars of the current case. In another alternatively, each path may be labeled with the number of cases resolved by that path.

Finally, a user interface (UI) is provided. In one embodiment, the UI could display the entire augmented diagnostic procedure with all available diagnostic paths labeled with the scores. However, this may be too complex for easy consumption by a time-pressed service engineer. In another (not necessarily mutually exclusive) embodiment, a ranked summary of the highest scoring paths is provided, perhaps with links to bring up the portions of the augmented diagnostic procedure including those highest-ranked paths.

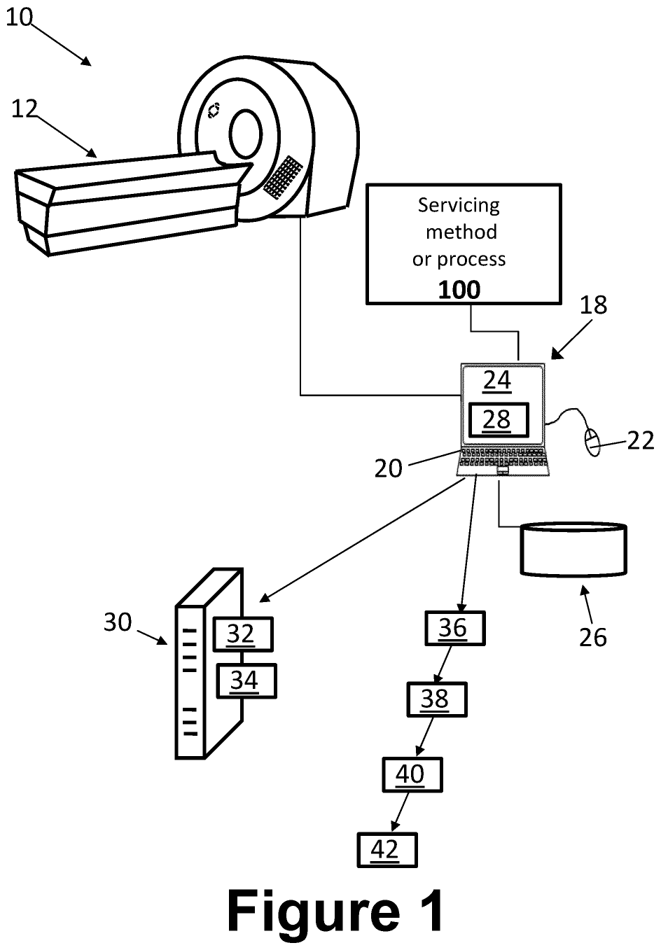

With reference to FIG. 1, an illustrative apparatus 10 for servicing a medical device 12 is shown. The medical device 12, for example, can comprise a medical imaging device 12 (also referred to as a medical device, an imaging device, imaging scanner, and variants thereof) can be a magnetic resonance imaging (MRI) scanner, a computed tomography (CT) scanner, a positron emission tomography (PET) scanner, a gamma camera for performing single photon emission computed tomography (SPECT), an interventional radiology (IR) device, an X-ray device, an image guided therapy (iGT) device, or so forth. Although shown in FIG. 1 as an imaging device, the medical device 12 can also be any other suitable medical device, such as a patient monitor, a radiation therapy device, a mechanical ventilator, and so forth.

An electronic processing device 18, such as a workstation computer, or more generally a computer, a smart device (e.g., a cellular telephone (“cell phone”), a smart tablet, and so forth), is operable by a service engineer (SE). The electronic processing device 18 may also include a server computer or a plurality of server computers, e.g., interconnected to form a server cluster, cloud computing resource, or so forth, to perform more complex computational tasks. The electronic processing device 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The electronic processing device 18 is also in communication with a database 30 (shown in FIG. 1 as a server computer) that stores service manuals 32 related to diagnostic procedures for a plurality of different medical devices (including the medical device 12 shown in FIG. 1). The database 30 also stores historical service records 34 related to previously-performed diagnostic procedures for diagnosing various malfunctions for a plurality of different medical devices (possibly including the medical device 12 shown in FIG. 1).

The apparatus 10 is configured as described above to perform a medical device servicing method or process 100. The non-transitory storage medium 26 stores instructions which are readable and executable by the at least one electronic processor 20 to perform disclosed operations including performing the servicing method or process 100. In some examples, the method 100 may be performed at least in part by cloud processing.

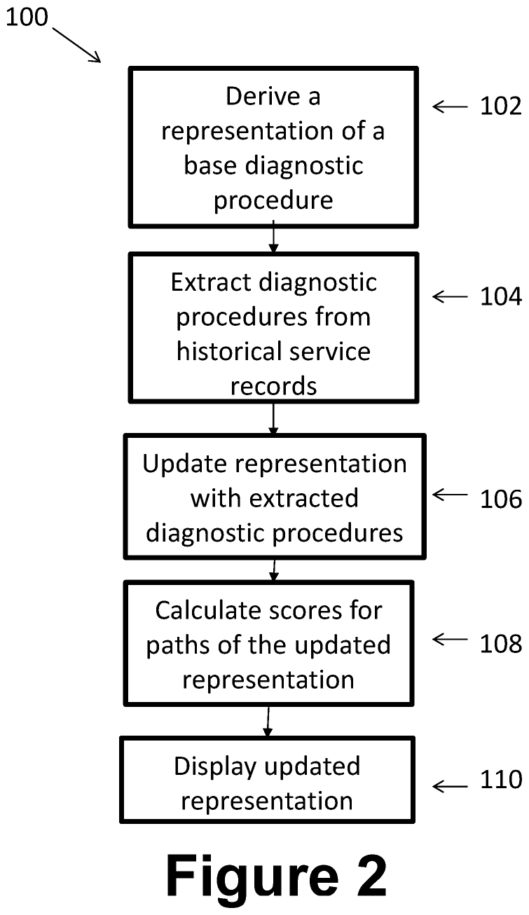

With reference to FIG. 2, and with continuing reference to FIG. 1, an illustrative embodiment of an instance of the servicing method 100 is diagrammatically shown as a flowchart. At an operation 102, a representation 36 of a base diagnostic procedure for diagnosing a malfunction of the medical device 12 is derived from one or more service manuals 32 retrieved from the database 30. The malfunction may be recognized in terms of specific symptoms, one or more error messages, observed deficiencies in the operation of the medical device 12, failure of a component of the medical device 12 to operate at all, and/or so forth. The base diagnostic procedure includes information such as problem symptoms indicative of the malfunction, potential root cause of the malfunction, error code(s) shown in a user interface, error code(s) logged in system (i.e., debug) log files, diagnostic steps to take to troubleshoot the problem, including expected outcomes, service actions to take to resolve the problem, and so forth. The representation 36 of the base diagnostic procedure can include, for example, a diagnostic tree, a diagnostic map, or any other suitable representation.

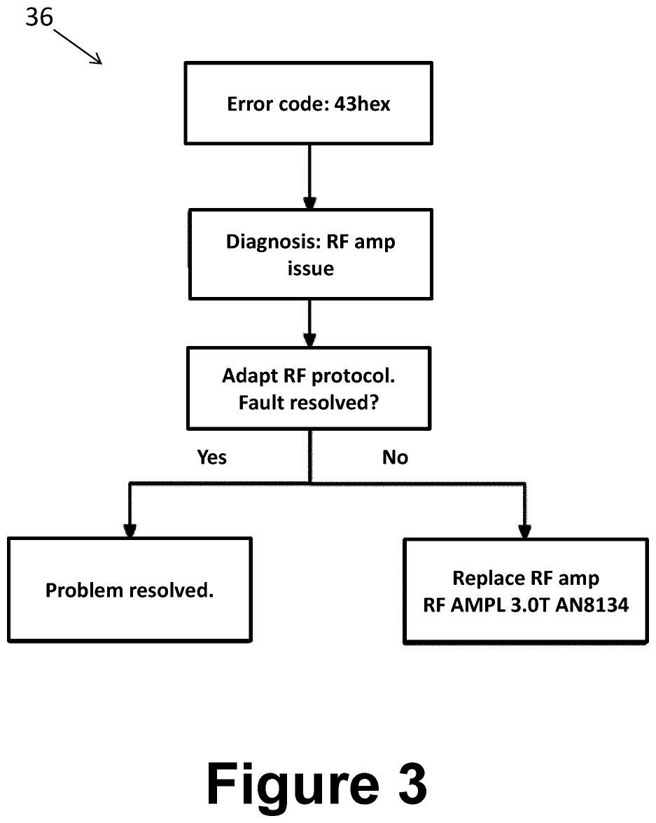

To derive the representation 36, a suitable process can be performed on the service manual(s) 32, such as natural language processing (NLP) process (i.e., when the information in the service manual(s) 32 is textual), a computer vision (CV) process (i.e., when the information in the service manual(s) 32 is graphical), manually, and so forth. For example, for an error code (e.g., “43hex” as one nonlimiting illustrative example) can be associated with two possible outcomes: either a scan protocol used is incorrect, or a component (i.e., a radiofrequency (RF) amplifier) is broken. FIG. 3 shows an example of the representation 36 of a base diagnostic procedure for an error code 43hex.

In some other embodiments, the representation 36 of the base diagnostic procedure for diagnosing the malfunction of the medical device can be provided directly, rather than being derived from service manual(s). For example, the representation 36 of the base diagnostic procedure may be provided in a directly consumable electronic form such as a data structure representing the base diagnostic procedure as fault tree. In such embodiments, the operation 102 is suitably omitted, or alternatively may be replaced by a retrieval operation that retrieves the data structure representing the fault tree.

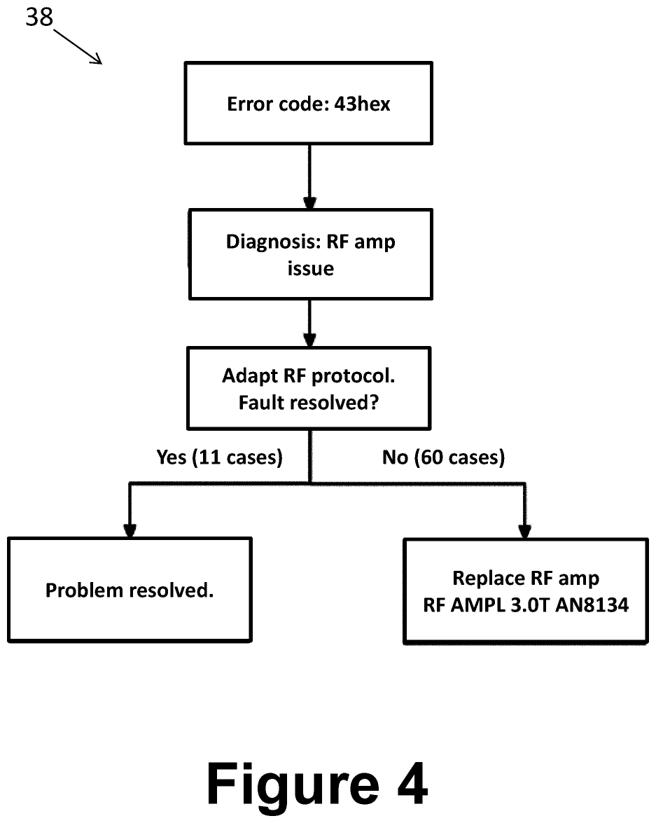

At an operation 104, one or more historical service records 34 (retrieved from the database 30) are analyzed to extract diagnostic procedures 38 applied in historical service cases for diagnosing the malfunction of the medical device 12. To extract the diagnostic procedures 38, a suitable process can be performed on the historical service record (s) 34, such as natural language processing (NLP) process, a computer vision (CV) process, and so forth. In a typical scenario, the historical service records 34 are in the form of structured service reports for historical service cases handled by service engineers. In this scenario, the relevant content may be extracted from structured and/or freeform fields of the service reports, such as a “service actions” field, a “diagnostic tests” field, and/or so forth. Real-time statistics on a number of times each procedure was used, what was the success rate of each taken step, which procedures were explored at the same time, and so forth, can be determined from the number of historical service records 34 that follow each procedure and the results indicated in the historical service records 34. FIG. 4 shows an example of the diagnostic procedure 38 for the error code 43hex.

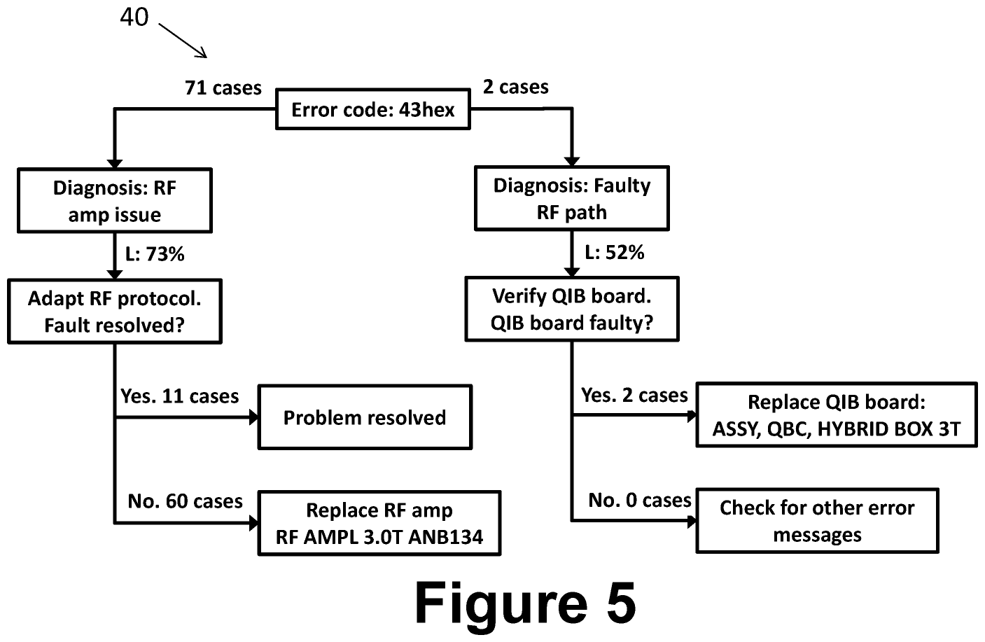

At an operation 106, the extracted diagnostic procedures are added to the representation 36 of the base diagnostic procedure to generate an updated representation 40 of the base diagnostic procedure. For example, common nodes of the extracted diagnostic procedures and the base diagnostic procedure are mapped, and nodes of the extracted diagnostic procedures that are not contained in the base diagnostic procedure are added. Furthermore, each path of the updated representation 40 of the base diagnostic procedure are labelled with a number of historical service cases 34 resolved by that path (or, in another embodiment, with a likelihood of success computed based on that path). FIG. 5 shows an example of the updated representation 40 of the base diagnostic procedure for the error code 43hex with the numbers of historical service cases 34 annotated to the paths. In some embodiments, the updated representation 40 can include adding additional paths described in number of historical service cases 34.

In some other embodiments, the representation 36 of the base diagnostic procedure is omitted entirely, and the operation 106 combines the extracted diagnostic procedures to generate a fault tree for diagnosing the malfunction of the medical device. In these embodiments, the fault tree constructed by combining the extracted diagnostic procedures (without starting from a base diagnostic procedure) corresponds to the updated representation 40 of the base diagnostic procedure in the subsequent operations.

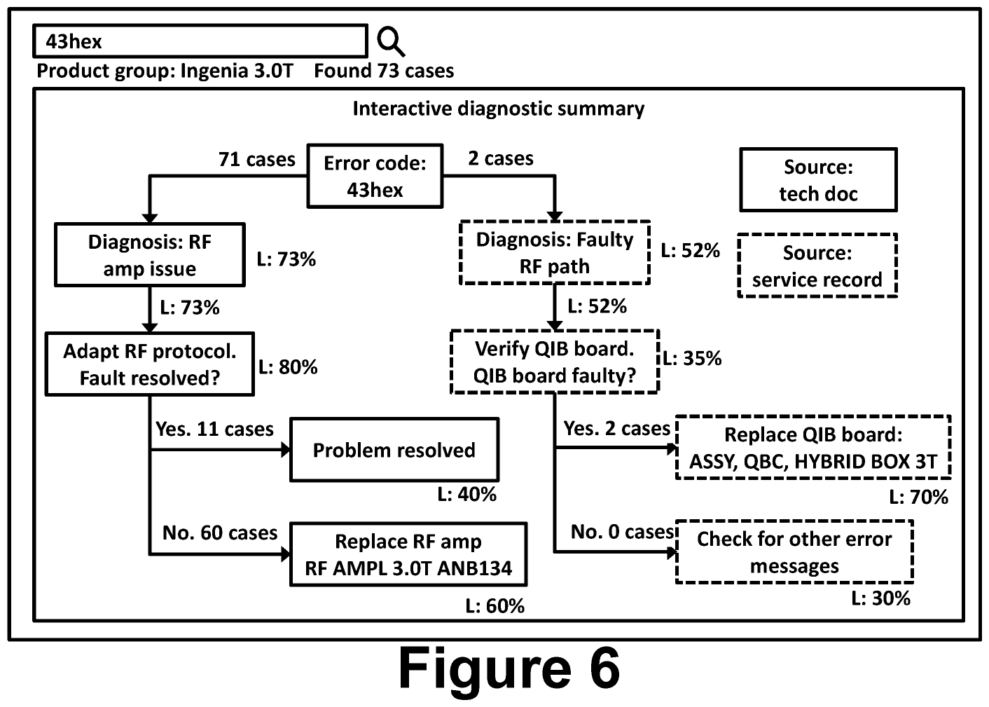

At an operation 108, likelihood of success scores 42 are calculated for each path of the updated representation 36 of the base diagnostic procedure based on statistics of outcomes of the historical service cases 34. (As previously noted, these can be used in place of the number of historical cases as the annotations for the updated representation 36). The likelihood of success scores 42 can indicate how likely that a service action will lead to successful service resolution for the given malfunction reported by the customer. FIG. 6 shows an example of the updated representation 40 of the base diagnostic procedure with the calculated likelihood of success scores 42 annotated to the paths for the updated representation 36 in which the error code 43hex is an observed symptom of the malfunction.

The likelihood of success scores 42 can be calculated based on one or more factors, including a number of historical service cases resolved by each path of the updated representation 40 of the base diagnostic procedure, outcome quality statistics of historical service cases 34 resolved by each path of the updated representation 40 of the base diagnostic procedure, a similarity metric of each path of the updated representation 40 of the base diagnostic procedure relative to the current service case, and so forth. In another embodiment, the likelihood of success scores 42 can be calculated based on a weighted sum of these three described factors.

In a particular example, given a problem p, a set of related cases C, a service action sᵢ and a set of cases where that service action was performed, Cₛ_{ᵢ}, the score can be calculated according to Equation 1:

$$score ( {s}_{i} , p ) = {w}_{1} \frac{\vert {C}_{{s}_{i}} \vert}{\vert C \vert} + {w}_{2} {\sum }_{{c}_{j} ∈ {C}_{{s}_{i}}} q ( {c}_{j} ) + {w}_{3} sim ( {s}_{i} , p ) \qquad ( 1 )$$

where w₁, w₂ and w₃ are weights for each factor described previously, q is a function that determines the quality-of-service delivery for a given case, and sim(sᵢ, p) is a similarity indicator of the problem description to the service action.

At an operation 110, the updated representation 40 of the base diagnostic procedure is output on, for example, on the display device 24 of the electronic processing device 18 via the GUI 28. In some embodiments, the displayed updated representation 40 can include the updated representation 40 with the likelihood of success scores 42 annotating the respective paths. In other embodiments, the displayed updated representation 40 can include the updated representation 40 as a ranked summary of paths having high likelihood of success scores 42.

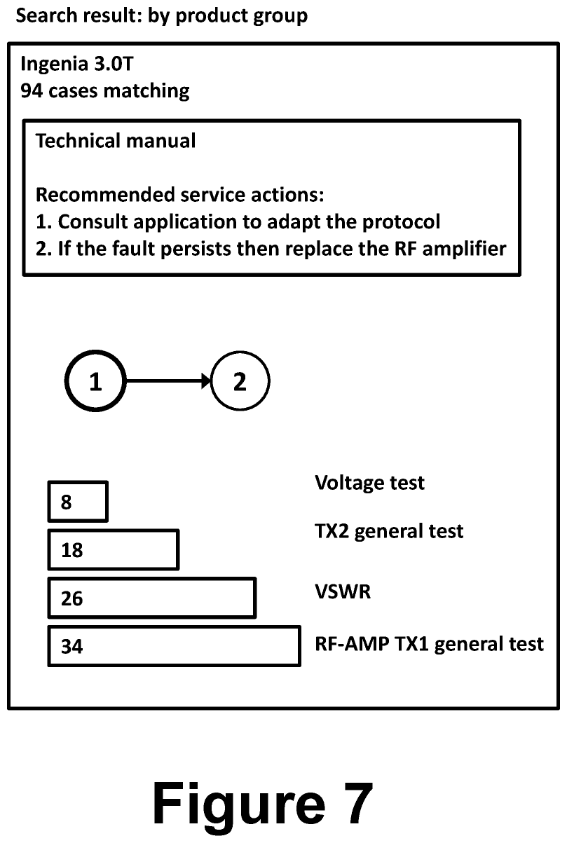

In some embodiments, as shown in FIG. 7, the displayed updated representation 40 can comprise a diagnostic tree (or can comprise nay other suitable representation such as a map with a bar chart). The RSE can then search the displayed updated representation 40 for an error code (i.e., 43hex). Referring back to FIG. 6, the updated representation 40 shows the result from both technical documentation (labelled in white rectangles) and service records (labelled in grey rectangles). The labels such as ‘L: 73%’ and ‘L: 52%’ indicate the corresponding likelihood of success. In this way, the RSE can see the source of information, as well as the predicted success rate. Optionally, if RSEs request the details of a certain action, then the RSEs can click it. From there, RSEs can navigate to the corresponding parts of the documentation, inspect the associate service cases, or trigger an analysis of the available log data from the medical device 12.

Once the RSE finishes following certain actions from the interactive diagnostic procedure, and assuming they the selected actions are selected from the displayed updated representation 40, a service record report can be automatically generated for the RSE for verification and stored as a historical service record 34 in the database 30.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a medical device servicing method for a medical device, the method comprising:
deriving a representation of a base diagnostic procedure for diagnosing a malfunction of the medical device from one or more service manuals;
analyzing historical service records to extract diagnostic procedures applied in historical service cases for diagnosing the malfunction of the medical device;
adding the extracted diagnostic procedures to the representation of the base diagnostic procedure to generate an updated representation of the base diagnostic procedure;
calculating likelihood of success scores for each path of the updated representation of the base diagnostic procedure based on statistics of outcomes of the historical service cases; and
outputting the updated representation of the base diagnostic procedure on an electronic processing device operable by a service engineer (SE).

2. The non-transitory computer readable medium of claim 1, wherein adding the extracted diagnostic procedures to the representation of the base diagnostic procedure to generate an updated representation of the base diagnostic procedure includes:
labelling each path of the updated representation of the base diagnostic procedure with a number of historical service cases resolved by that path.

3. The non-transitory computer readable medium of claim 1, wherein adding the extracted diagnostic procedures to the representation of the base diagnostic procedure to generate an updated representation of the base diagnostic procedure includes:
adding additional paths described in the historical service records to the updated representation.

4. The non-transitory computer readable medium of claim 1, wherein calculating likelihood of success scores for each path of the updated representation of the base diagnostic procedure includes:
calculating the likelihood of success scores based on a number of historical service cases resolved by each path.

5. The non-transitory computer readable medium of claim 1, wherein calculating likelihood of success scores for each path of the updated representation of the base diagnostic procedure includes:
calculating the likelihood of success scores based on outcome quality statistics of historical service cases resolved by each path.

6. The non-transitory computer readable medium of claim 1, wherein the likelihood of success scores are calculated with respect to a current service case and calculating likelihood of success scores for each path of the updated representation of the base diagnostic procedure includes:
calculating the likelihood of success scores based on a similarity metric of each path relative to the current service case.

7. The non-transitory computer readable medium of claim 1, wherein analyzing historical service records to extract diagnostic procedures applied in historical service cases for diagnosing the malfunction of the medical device includes:
performing a natural language processing (NLP) process on the historical service records.

8. The non-transitory computer readable medium of claim 1, wherein outputting the updated representation of the base diagnostic procedure on an electronic processing device operable by a SE includes:
displaying the updated representation with the likelihood of success scores annotating the respective paths.

9. The non-transitory computer readable medium of claim 1, wherein outputting the updated representation of the base diagnostic procedure on an electronic processing device operable by a SE includes:
displaying the updated representation as a ranked summary of paths having high likelihood of success scores.

10. The non-transitory computer readable medium of claim 1, wherein the representation of a base diagnostic procedure comprises one of a diagnostic tree or a diagnostic map.

11. The non-transitory computer readable medium of claim 1, wherein retrieving a representation of a base diagnostic procedure for diagnosing the malfunction of the medical device from one or more service manuals includes:
performing a natural language processing (NLP) process on the one or more service manuals.

12. The non-transitory computer readable medium of claim 1, wherein retrieving a representation of a base diagnostic procedure for diagnosing the malfunction of the medical device from one or more service manuals includes:
performing a computer vision (CV) process on the one or more service manuals.

13. The non-transitory computer readable medium of claim 1, wherein each path of the updated representation of the base diagnostic procedure comprises one or more routes or procedures performed by the SE for diagnosing a malfunction of the medical device.

14. An apparatus, comprising:
a display device; and
at least one electronic processor programmed to:
augment a representation of a base diagnostic procedure for diagnosing a malfunction of the medical device with diagnostic procedures applied in historical service cases for diagnosing the malfunction of the medical device to generate an updated representation of the base diagnostic procedure;
calculate likelihood of success scores for each path of the updated representation of the base diagnostic procedure based on statistics of outcomes of the historical service cases; and
output the updated representation of the base diagnostic procedure on the display device including at least a portion of the paths of the updated representation annotated by the calculated likelihood of success scores.

15. The apparatus of claim 14, wherein the likelihood of success scores are calculated as a weighted sum of a number of historical service cases resolved by each path, outcome quality statistics of historical service cases resolved by each path, and a similarity metric of each path relative to the current service case.

16. The apparatus of claim 14, wherein the at least one electronic processor is programmed to analyze historical service records to extract the diagnostic procedures applied in historical service cases for diagnosing the malfunction of the medical device.

17. The apparatus of claim 14, wherein the at least one electronic processor is programmed to output the updated representation of the base diagnostic procedure comprising:
displaying the updated representation as a ranked summary of paths having a highest likelihood of success scores with each path of the ranked summary annotated by the calculated likelihood of success score for that path.

18. The apparatus of claim 14, wherein the at least one electronic processor is further programmed to derive the representation of the base diagnostic procedure for diagnosing the malfunction of the medical device from one or more service manuals.

19. The apparatus of claim 18, wherein the at least one electronic processor is programmed to derive the representation of the base diagnostic procedure by operations including one or more of:
performing a natural language processing (NLP) process on the one or more service manuals; or
performing a computer vision (CV) process on the one or more service manuals.

20. A medical device servicing method for a medical device, the method comprising:
analyzing historical service records to extract diagnostic procedures applied in historical service cases for diagnosing a malfunction of the medical device;
combining the extracted diagnostic procedures to generate a fault tree for diagnosing the malfunction of the medical device;
labelling each path of the fault tree with a label comprising a number of historical service cases resolved by that path or with a likelihood of success score calculated based on the number of historical service cases resolved by that path, wherein each path comprises one or more routes or procedures performed by a service engineer for diagnosing a malfunction of the medical device; and
outputting a representation of the fault tree including the labels.
